(19) United States
(12) Patent Application Publication
(10) Pub. No.: US 2026/0315268 A1
(43) Pub. Date: Oct. 15, 2026

(54) SYSTEM FOR COLLECTING BODY WASTE FROM AN ANUS OF A USER

(71) Applicant: Furine ApS, Skævinge (DK)

(72) Inventor: Brian Thomsen NIELSEN, Frederikssund (DK)

(73) Assignee: Furine ApS, Skævinge (DK)

(21) Appl. No.: 19/483,888

(22) PCT Filed: Jun. 7, 2024
(86) PCT No.: PCT/EP2024/065839

§ 371 Date: Nov. 12, 2025

(30) Foreign Application Priority Data
Jun. 7, 2023 (DK) PA202370274
Jun. 21, 2023 (DK) PA202370328

(51) Int. Cl. A61F 5/443

(52) U.S. Cl. CPC A61F 5/443

(57) ABSTRACT

A system for collecting faecal matter from an anus of a user, said system comprising: an attachment member comprising an attachment flange comprising a skin friendly adhesive layer with a skin facing surface, an opening in the attachment flange which is in fluid communication with the anus, a bag engaging portion comprising a flexible flange and an opening in the flange, a tubular portion between the attachment flange and the bag engaging portion, and a collection bag, said collection bag comprising a reservoir for collecting and storing faecal matter from the user, an opening which is in fluid communication with the reservoir, and a flexible surface surrounding the opening for detachably attaching the flexible surface of the collection bag to the flexible flange of the bag engaging portion of the attachment member via an adhesive.

The current invention relates to a system for collecting body waste from an anus of a user, said system comprising: an attachment member arranged to be attached to perianal skin surrounding the anus, said attachment member comprising an attachment flange which is arranged to be attached to said skin such that a fluid tight seal is established between the attachment flange and said skin entirely around the anus when the attachment member is attached to the user, said attachment flange comprising a skin friendly adhesive layer with a skin facing surface and an opening in the attachment flange which is in fluid communication with the anus when the attachment member is attached to the user.

For the sake of this specification, the term “body waste” should be interpreted as both faecal waste and urinary waste unless otherwise indicated.

It should be noted that the present specification discloses multiple inventions. A first invention, related to an inventive type of system for collecting body waste from a user is defined in the claim set. A second invention related to a first inventive arrangement of an attachment flange of a system for collecting body waste from a user is defined by examples provided in the description. A third invention related to a second inventive arrangement of an attachment flange of a system for collecting body waste from a user is also defined by examples in the description. A fourth invention related to an inventive type of hybrid system for collecting body waste from a user is also defined by examples in the description. Divisional applications directed to the second, third and/or fourth inventions could be filed in the future. Other inventions are also disclosed in this specification as examples of alternative arrangements of the attachment member. These other inventions could also be the subject of potential divisional applications in the future.

It should also be noted that comments with regards to understanding of this specification made in respect to one invention, should also read on the other inventions unless otherwise specifically mentioned. Likewise, examples or features of embodiments provided with regards to one specific invention, should also be applicable to the other inventions, if the person skilled in the art would consider the features to be relevant for the other inventions based on the teachings of this specification.

### DESCRIPTION OF RELATED ART

Prior art systems for collecting body waste from a user are well known in the art. Some relevant examples are disclosed in applicant's own pending applications, see especially WO2018/050856, WO2019/179586 and WO2020/182923 which are incorporated by reference in this application.

The above-mentioned systems are all advantageous with regards to even more prior applications, however, the current inventions disclosed in the current specification provide additional features which in certain cases provide improved systems.

### SUMMARY OF THE INVENTION

A first aspect of the first invention is to provide a system which is more securely attached to the user to ensure a low risk of leakage.

A second aspect of the first invention is to provide a system which is more comfortable for the user to wear.

A third aspect of the first invention is to provide a system which is easy to use for the caretaker.

A fourth aspect of the first invention is to provide a system which works with hard stool in addition to more liquid stool.

A fifth aspect of the first invention is to provide a system where the collection bag can be replaced in a simple manner and in a manner which is less uncomfortable to the user as compared to prior art systems.

At least some of these aspects are provided according to a system as according to claim 1. In this way, a system is provided whereby a collection bag can be attached to a bag engaging portion of an attachment member which is attached to the perianal skin of a user in an easy way. Furthermore, since the flexible flange of the bag engaging portion is arranged to be folded in half by the buttocks of the user, the flexible flange and also the flexible surface of the collection bag can be arranged between the buttocks of the user during normal use.

It should be noted that according to the current claim 1, the bag engaging portion comprises a tubular portion between the attachment flange and the flexible flange of the bag engaging portion. However, in other examples, it could be imagined that the flexible flange of the bag engaging portion could be attached directly to the attachment flange via an annular or ring formed connection. In such examples, the annular connection could be arranged such that a dimension D4 of a first vector connecting the outermost points of said annular connection in a sagittal plane of a user when connected to a user, is greater than 20 mm and less than 80 mm. In some such examples, the bag engaging portion could be arranged such that a distance D6, in the sagittal plane of a user when connected to a user, from an innermost portion of the annular connection to an innermost portion of the bag engaging portion arranged furthest from the annular connection along a direction which is perpendicular to the first vector, is less than 3 cm or less than 1 cm.

In some embodiments, the tubular portion is arranged such that a dimension D4 of an “opening” vector connecting the outermost points of the ring formed connection between the attachment flange and the tubular portion in a sagittal plane of a user when connected to a user, is greater than 20 mm and less than 80 mm.

It should be noted that in this specification, terms in the claims which are in quotes, are used to identify an element/portion of a device, but are not used to define the element/portion. For example the term “opening” when used as “opening” vector, should be understood in the same way as if “first” vector had been used. The term “opening” should not be given structural meaning, but only be used for identification.

In some embodiments, the bag engaging portion, the tubular portion and the attachment flange are arranged such that a dimension D6a, in the sagittal plane of a user when connected to a user, from an outermost portion of the ring formed connection to an outermost portion of the connection between the tubular portion and the bag engaging portion arranged furthest from the ring formed connection along a direction which is perpendicular to an “opening” vector connecting the outermost points of the ring formed connection in a sagittal plane of a user when connected to a user is less than the minimum radial dimension along the surface of the attachment flange from the centre of the opening in the attachment flange to the outer periphery of the attachment flange.

It should be noted that in this specification, the “outermost points of the ring formed connection” should be understood as the points of the ring formed connection which are furthest from the anus of the user when the attachment member is attached to a user. Likewise, the “innermost points of the ring formed connection” would be understood as the points of the ring formed connection which are closest to the anus of the user when the attachment member is attached to a user. In many of the current embodiments, the wall thickness of the tubular portion is very small, and hence, the innermost points and outermost points of the ring formed connection are almost the same.

It should also be noted that the distance D6a is defined as the distance between an outermost portion of the ring formed connection (between the attachment flange and the tubular portion) and an outermost portion of the connection between the tubular portion and the bag engaging portion. Again, the term “outermost” in this case refers to the distance from the anus of the user. In the case where the tubular portion has a small wall thickness, the outermost portion of the ring formed connection and the innermost portion of the ring formed connection are almost the same. In these situations, it would also be possible to just refer to the distance D6 as the distance between the ring formed connection and the connection between the tubular portion and the bag engaging portion. In other cases, it could also be suitable to define the distance D6 as the “length” of the tubular portion.

In some embodiments, the tubular portion is less than 5 cm, 3 cm, 1 cm, 0.75 cm, 0.5 cm or 0.35 cm long. In some embodiments, the distance D4 is greater than 25 mm, greater than 30 mm or greater than 35 mm. In some embodiments, the distance D4 is less than 70 mm, less than 60 mm or less than 50 mm.

In some embodiments, the bag engaging portion, the tubular portion and the attachment flange are arranged such that a dimension D6a, in the sagittal plane of a user when connected to a user, from an outermost portion of the ring formed connection to an outermost portion of the connection between the tubular portion and the bag engaging portion arranged furthest from the ring formed connection along a direction which is perpendicular to an “opening” vector connecting the outermost points of the ring formed connection in a sagittal plane of a user when connected to a user is greater than a dimension D6b in a plane which is perpendicular to said “opening” vector, from an outermost portion of the ring formed connection to an outermost portion of the connection between the tubular portion and the bag engaging portion arranged furthest from the attachment flange along a direction which is perpendicular to the “opening” vector.

In some embodiments, the bag engaging portion, the tubular portion and the attachment flange are arranged such that a dimension D6a, in the sagittal plane of a user when connected to a user, from an outermost portion of the ring formed connection to an outermost portion of the connection between the tubular portion and the bag engaging portion arranged furthest from the ring formed connection along a direction which is perpendicular to an “opening” vector connecting the 20 outermost points of the ring formed connection in a sagittal plane of a user when connected to a user and/or a dimension D6b, in a “perpendicular” plane which is perpendicular to an “opening” vector connecting the outermost points of the ring formed connection in a sagittal plane of a user when connected to a user, from an outermost portion of the ring formed connection to an outermost portion of the connection between the tubular portion and the bag engaging portion arranged furthest from the attachment flange along a direction which is perpendicular to the “opening” vector are/is less than the radial dimension along the surface of the attachment flange from the centre of the opening in the attachment flange to the outer periphery of the attachment flange along said “perpendicular” plane.

In some embodiments, the attachment member comprises holding means to hold the opening of the bag engaging portion essentially aligned with the opening in the attachment flange. In some embodiments, the holding means are arranged to hold the rim of the bag engaging portion essentially perpendicular to a centre axis passing through the tubular portion and/or the opening.

In some embodiments, the opening of the bag engaging portion is essentially aligned with the opening in the attachment flange. In some embodiments, the opening in the attachment flange and the opening in the flexible flange of the bag engaging portion are arranged essentially co-axially. In some embodiments, a centre axis of the opening in the attachment flange would pass through the opening in the bag engaging portion.

In some embodiments, the ring formed connection has a minimum internal diameter which is greater than 30 mm. According to this specification, the phrase “minimum internal diameter” should be understood as the minimum dimension from one side of the ring formed connection to the other side of the ring formed connection, when passing through the centre axis of the tubular portion. In some embodiments, the tubular portion and/or the ring formed connection has a non-circular cross section. In some embodiments, the ring formed connection and/or the tubular portion of the bag engaging portion has a cross sectional area on a plane which is parallel to the attachment flange which is greater than 5 cm², 8 cm², 10 cm² or 12 cm².

In some embodiments, the outermost 5 mm of the attachment flange and the outermost 5 mm of the flexible flange of the bag engaging portion are arranged such that the two portions can displace relative to each other. In some embodiments, the outer 7 mm, 8 mm or 9 mm of the attachment flange and flexible flange of the bag engaging portions can displace relative to each other.

In some embodiments, the attachment flange has an outer diameter which is larger than an outer diameter of the ring formed connection. In some embodiments, the flexible flange of the bag engaging portion has an outer diameter which is greater than the outer diameter of the ring formed connection. In some embodiments, the attachment flange and/or the flexible flange of the bag engaging portion is more than 5 mm, 7 mm or 10 mm greater than the ring formed connection.

In some embodiments, the flexible flange of the bag engaging portion is displaced from the outer circumference of the attachment flange.

In some embodiments, the attachment flange is made of a stretchable elastic material while the engaging portion of the collection bag is made of an essentially plastic material. For example non TPE. In some embodiments, stretchable elastic material means having an elongation at break of greater than 100%, greater than 150% or greater than 200%. In some embodiments, the stretchable elastic material means having an elongation per unit force which is at least 150%/Newton for a 15 mm wide strip of the material. The stretchability of the portion of the attachment flange between the outermost portion of the ring formed connection and the outermost periphery of the attachment flange being greater than the stretchability of the bag engaging portion.

In some embodiments, an angle A between a “skin facing surface” vector which is parallel with the skin facing surface of the adhesive layer of the attachment flange and a “tubular portion” vector which is parallel to an inner surface of the tubular portion is greater than 70 degrees. Note that the angle is defined on an inner portion of the tubular portion. In this way, it is easy to clean the inner surface of the tubular portion when switching collection bags. In some embodiments, the angle A is defined on a plane which is parallel to an “opening” vector which is perpendicular to the first vector and on the sagittal plane of a user when connected to a user, In some embodiments, the angle A in all planes which are parallel to the “opening” vector is greater than 70 degrees. In some embodiments, the angle A is greater than 80 degrees, greater than 90 degrees or greater than 100 degrees. If the tubular portion does not define a straight path, then the “tubular portion” vector is defined as the average tangent vector along the length of the tubular portion. Likewise, if the skin facing surface does not define a straight path, then the “skin facing surface” vector is defined as the average tangent vector to the skin facing surface.

In some embodiments, the inner diameter of the opening in the flexible flange of the bag engaging portion is greater than or equal to the inner diameter of the tubular portion at a ring formed connection between the tubular portion and the flexible flange of the bag engaging portion. Again, this ensures easy cleaning of the tubular portion during a bag switch. Likewise, in certain embodiments, the inner diameter of the opening in the flexible flange of the bag engaging portion is greater than or equal to the inner diameter of the opening in the attachment flange.

In some embodiments, the opening in the attachment flange is oval. In certain cases, this can optimize the shape of the attachment member and effectively attach it to the peri-anal skin.

In some embodiments, the attachment flange comprises a first portion having an inner periphery and an outer periphery arranged spaced away from the inner periphery in a direction away from the opening in the attachment flange and a second portion extending between the outer periphery of the first portion and an outer periphery of the second portion which is further away from the opening in the attachment flange than the outer periphery of the first portion, said first portion having an elongation per unit force applied along the attachment flange and said second portion having a different elongation per unit force applied along the attachment flange which is higher than the elongation per unit force of the first portion. It should be clear that the different features of the “second invention” disclosed in this specification could be added to this claim as additional features.

In certain embodiments, the elongation per unit force of the second portion is at least 150%/Newton, at least 200%/Newton or at least 250%/Newton for a 15 mm wide strip of material of the second portion.

In some embodiments, the attachment flange comprises a central portion extending from the inner periphery of the first portion towards the opening in the attachment flange, said central portion having an elongation per unit force applied along the attachment flange which is higher than the elongation per unit force of the first portion. In some embodiments, the elongation per unit force along the attachment flange of the central portion is at least 2, 4, 6 or 8 times greater than the elongation per unit force of the first portion. In some embodiments, the elongation per unit force of the central portion is similar to the elongation per unit force of the second portion. In some embodiments similar elongation per unit force values should be values which are within 3, 2 or 1.5 times each other. It should be clear that the features of the third invention disclosed in this specification could be added to this claim as additional features.

In certain embodiments, the elongation per unit force of the central portion is at least 150%/Newton, at least 200%/Newton or at least 250%/Newton for a 15 mm wide strip of material of the central portion.

The current specification also discloses a second invention as mentioned above.

A first aspect of the second invention is to provide a system which is more securely attached to the user to ensure a low risk of leakage.

A second aspect of the second invention is to provide a system which is more comfortable for the user to wear.

A third aspect of the second invention is to provide a system which is easy to use for the caretaker.

At least some of the aspects are provided according to a system as mentioned in the introductory paragraph of example 1 below where the attachment flange comprises a first portion extending between an inner periphery of the first portion and an outer periphery of the first portion arranged spaced away from the ring formed connection and away from the opening in the attachment flange and a second portion extending between the outer periphery of the first portion and an outer periphery of the second portion which is further away from the opening in the attachment flange than the outer periphery of the first portion, said first portion having an elongation per unit force applied along the attachment flange and said second portion having a different elongation per unit force applied along the attachment flange which is higher than the elongation per unit force of the first portion. In this way, the first portion can provide a strong connection to the tubular portion and the second portion can provide a strong and flexible connection to the user. By allowing the second portion to be softer and more stretchable than the first portion, the tensions of the first portion and not transferred all the way to the outer parts of the second portion. This provides for a more secure connection. In some embodiments, the inner periphery of the first portion is arranged at an inner periphery of the ring formed connection. In some embodiments the outer periphery of the first portion is arranged at an outer periphery of the ring formed connection.

It should be noted that the tubular portion of the attachment member can be connected to a collection bag placed close to the user or far away from the user via a longer tubular conduit. In certain cases, the tubular portion is provided with a connector which can connect to a collection bag or a longer conduit. In other cases, the tubular portion is an integrated part of a longer tubular conduit which is then connected to a collection bag, either directly or via a connector. In certain cases, the length of the tubular portion can be very short. For example, an opening of a collection bag could be connected essentially directly to the ring formed connection. In this case, the “tubular portion” would be essentially non-existent. However, for the sake of this specification, in such a situation, one could still consider the presence of a tubular portion with a very short length and having a first end and a second end, a first end being connected to the attachment flange via a ring formed connection and a bag engaging portion formed at the second end of the very short tubular portion. In general, the tubular portion is a portion which connects the opening in the attachment flange and the collection bag. The length of the tubular portion can vary significantly depending on the application and embodiment.

It should be noted that in this specification, the phrase “elongation per unit force along the attachment flange” should be interpreted as the property of the attachment flange to stretch so that its width increases. In this specification the term width of a portion is defined as the distance from the inner periphery of the portion to the outer periphery of the portion, along a vector passing through the centre of the opening. The attachment flange can also stretch perpendicular to the plane of the flange, but different layers of the flange could stretch different amounts perpendicular to the plane of the flange. In contrast, when stretching in the plane of the flange, the different layers of the flange will stretch together and the different layers will therefore have a significant impact on the overall stretchability of the flange. In addition, the material properties and the dimensions of the different layers will contribute to the specific elongation of the flange when a force is applied to the flange. This is described in more detail in the detailed description.

It should also be noted that there is a difference between the terms flexible and stretchable/elastic. A well-known plastic bag is flexible as it can adapt to many shapes by bending, however, it is not very stretchable or elastic. If you stretch it, it will stretch permanently and be damaged. If you stretch it enough it will break. An elastic or stretchable material will for normal stretches be elastic and return to its original shape after releasing the force used to stretch it. The attachment flanges in the example embodiments are both flexible and elastic/stretchable.

In some embodiments, the elongation per unit force of the second portion is at least 2, 4, 6 or 8 times greater than the elongation per unit force of the first portion. In case the material properties and/or the dimension of the attachment flange change along the length of the attachment flange, then the elongation per unit force at different locations on the flange will be different. However, for the sake of interpreting the claims, the different portions will still have an elongation per unit force, even though the actual amount of stretch at different locations in a single portion might be different. For example if the flange member tapers from the inside to the outside, then the outer portion will stretch more than the inner portion. It can also be noted that in certain embodiments, the portions of the attachment flange might blend into each other, making it difficult to specify an exact end of the first portion. In this case, an arbitrary position can be chosen for the end of the first portion and an arbitrary start of the second position can be chosen outside the end of the first portion. See for example, the description associated with FIG. 9.

It should be noted that in this specification, specific details of the adhesives used in the different inventions are not provided. However, different suitable adhesives are available in the art. For more specific details of one suitable adhesive, the reader is referred to applicant's own prior publication WO2019/179586 which discloses a number of suitable adhesives. It should be noted that in particular, a two component adhesive, as also described in WO2019/179586, is especially suitable for the current invention. In one particular suitable two component adhesive, a first thin adhesive layer is first applied to the skin of the user after which the attachment flange with the second adhesive layer is applied on the first thin adhesive layer.

In this specification, the term “ring formed connection” should be understood as a connection between the attachment flange and the tubular portion and/or connection portion. The attachment flange and the tubular portion and/or connection portion could be separate elements which are connected via an adhesive or via a welding operation. Or the attachment flange (or a portion of the attachment flange) and the tubular portion/connection portion (or a portion of the tubular portion/connection portion) could be two portions of a single element, for example an injection moulded element. In this case, the connection between the attachment flange and the tubular/connection portion is formed via the integral formation of the attachment flange and the tubular/connection portion. In general, the attachment flange will be considered the portion which is adhered to the skin of the user via the adhesive on the attachment flange and the tubular/connection portion will be the portion which is extending away from the attachment flange and away from the skin of the user. The ring formed connection will be where the attachment flange and the tubular/connection portion meet. Different examples and further discussions on this definition will be provided with respect to concrete embodiments in the detailed description below. It should also be noted that “ring formed” includes non-circular shapes, such as oval, square, undulating, etc. The shape itself is not limiting for the scope of protection.

In some embodiments, the first portion is at least 2 mm wide and the second portion is at least 5 mm wide. In some embodiments, the first portion is at least 3 mm or at least 4 mm or at least 5 mm wide. In some embodiments, the second portion is at least 10 mm, at least 15 mm or at least 20 mm wide. It should be noted that the width of the portion does not have to be constant around the circumference of the attachment flange, but could vary. In case the width is not constant, then the average width of the portion can be used with regards to claims specifying the width of the portion.

In some embodiments, the first portion comprises a flange member which is connected to the tubular portion at the ring formed connection and an adhesive layer arranged on the skin facing surface of the flange member and in that the second portion comprises an adhesive layer with a skin facing surface and a backing layer on the surface of the adhesive layer facing away from the skin, said backing layer being less adhesive than the adhesive layer. By backing layer should be understood a layer which prevents adhesiveness. This could for example, be a layer of talc, Calcium Carbonate, etc. In other words, the backing layer could be a powder that is attached to eliminate the adhesion without significantly altering the stretchability of the adhesive layer. Or a very thin film could be painted or sprayed onto the rear surface of the adhesive layer. In another embodiment, a film could be applied which is afterwards cut up into many small pieces such that the film layer does not have significant effect on the stretchability. In other embodiments, the backing layer and the flange member could be combined into a single integrated element, but where the outer portion of the element is much thinner or much more stretchable than the inner portion of the element. This could for example be provided via a tapering flange member. In other cases, the backing layer is a thin layer of elastic film onto which the adhesive layer is provided.

In some embodiments, the adhesive layer of the first portion and the adhesive layer of the second portion form a single connected adhesive layer. In some embodiments, the thickness of the adhesive layer could be greater than 1 mm, 2 mm or 3 mm.

In some embodiments, the backing layer of the second portion extends into the first portion. In some embodiments, where the backing layer extends into the first portion, the backing layer in the first portion could be arranged between the flange member and the adhesive layer.

In some embodiments, the first and second portions of the attachment flange comprise an adhesive layer which is applied on a backing layer, and in that the adhesive layers of the first and second portions are part of a single connected adhesive layer and in that the backing layers of the first and second portions are part of a single connected backing layer. In some embodiments, the flange member is attached to the backing layer in the first portion of the attachment flange.

In some embodiments, the backing layer could be a PU-film, a silicone film, or a similar elastic film. The backing layer could have a thickness which was less than 0.1 mm, 0.05 mm, 0.03 mm or 0.01 mm. The backing layer could have a shore hardness which is less than Shore A20, A10, A5 or 0035.

In some embodiments, the adhesive layer of the first portion and the adhesive layer of the second portion have a shore harness of Shore A: 10 or less. By making the adhesive have a low hardness, the adhesive will be able to stretch and adapt to the user's movements and shape in a comfortable and secure way. In some embodiments the adhesive layers have a shore hardness of Shore A: 5 or less, Shore A: 2 or less, Shore 00: 30 or less, Shore 00: 20 or less or Shore 000: 35 or less.

In some embodiments, the backing layer has an elongation per unit force along the attachment flange which is larger than the elongation per unit force along the attachment flange of the flange member. By making the backing layer more stretchable than the flange member, the backing layer will not transfer the forces from the tubular portion to the end of the attachment flange in the same way as if it had had the same stretchability as the flange member.

In some embodiments, the elongation per unit force along the attachment flange of the backing layer is at least 4, 8, 10 or 15 times larger than the elongation per unit force along the attachment flange of the flange member. In some embodiments, the backing layer does not apply any significant stress to the adhesive layer of the second portion when the tubular portion is pulled away from the attachment flange. In some embodiments, the exposed surface of the backing layer is not adhesive. In some embodiments, the backing layer extends over at least a portion of the first layer. In some embodiments, the flange member and/or the tubular portion is made of a non-adhesive material. In some embodiments, the elongation at break of the backing layer is less than the elongation at break of the flange member. In some embodiments, the elongation at break of the backing layer is equal to the elongation at break of the adhesive layer of the second portion or the elongation at break of the backing layer is less than 50% greater than the elongation at break of the adhesive layer of the second portion. In some embodiments, the elongation at break of the backing layer of the second portion is less than the elongation at break of the adhesive layer of the second portion. In this way, the backing layer can provide structure and stability to the adhesive layer. It can also protect the adhesive layer from damage.

In some embodiments, the second portion has a shore hardness of Shore A: 10 or less. For the sake of this specification, when an embodiment comprising an element is described as having a shore hardness below a certain level, and the element comprises multiple materials, then the shore hardness of all the materials should be less than the specified shore hardness. In some embodiments the second portion has a shore hardness of Shore A: 5 or less, Shore A: 2 or less, Shore 00: 30 or less, Shore 00: 20 or less or Shore 000: 35 or less.

In some embodiments, the attachment flange further comprises a central portion, said central portion extending from the inner periphery of the first portion towards the orifice when the attachment member is attached to the user, said first portion having an elongation per unit force applied along the attachment flange and said central portion having a different elongation per unit force applied along the attachment flange which is higher than the elongation per unit force of the first portion. By providing a “soft” and stretchable central portion, the central portion can adapt to geometry changes of the orifice. For example during stool movements, the anus will enlarge. The soft central portion will then adapt and enlarge as well. In this way, a strong connection can be established between the central portion and the perianal skin, but the skin can still adapt during stool movements. The same can be true around a stoma where the central portion can be pressed up against the stoma in a comfortable way, while still maintaining a good seal and allowing the stoma to change in geometry if necessary. In some embodiments, the elongation per unit force of the central portion is at least 2, 4, 6 or 8 times greater than the elongation per unit force of the first portion.

In some embodiments, the width of the central portion is at least 2 mm. In some embodiments, the width of the central portion is at least 4 mm, at least 6 mm or at least 8 mm.

In some embodiments, the adhesive layer of the first portion and the adhesive layer of the central portion form a single connected adhesive layer. In some embodiments, where there is a first, a second and a central portion, the adhesive layers of all three portions form a single connected adhesive layer.

In some embodiments, the central portion has a shore hardness of Shore A: 10 or less. In some embodiments, the central portion has a shore hardness of Shore A: 5 or less, Shore A: 2 or less, Shore 00: 30 or less, Shore 00: 20 or less or Shore 000: 35 or less.

In some embodiments, the attachment flange and the tubular portion are made of essentially non-water-absorbing materials. In this way, it is possible to wash and then reuse the attachment member. In some embodiments, said material could be Silicone, TPU, Elastomers, soft PVC, PU and other materials suitable for tubular extrusion or a combination of materials. In some embodiments, the attachment flange and the tubular portion are made of essentially non-ethanol absorbing materials. This makes it possible to wash the attachment member with alcohol without deteriorating the properties of the attachment member. Similarly, in some embodiments, the composition of the adhesive layer is chosen such that when the attachment flange is washed, the adhesive properties of the adhesive layer are not affected, or where the adhesive properties of the adhesive layer are not reduced by more than 20% after a typical washing operation.

It should be noted that most of the body waste collecting systems shown in the figures are designed to collect faecal waste from the anus of a user and are connected to the perianal skin of a user. However, certain inventions of the body waste collecting system as disclosed in this specification could also be used to collect faecal waste coming from a stoma of a user. Instead of mounting the attachment member to the perianal skin around the anus, the attachment flange could be adhered to the peristomal skin around the stoma of a user. As the stoma typically protrudes from the skin, the stoma can be arranged to fit inside the opening of the attachment member. In certain embodiments, an ostomy wax or ostomy ring could be applied between the stoma and the attachment member to further seal the connection between the stoma and the attachment member. This type of body waste collecting system could be attached to colostomies, ileostomies and urostomies and collect faecal matter or urinary matter.

In the figures, the attachment flange is shown having a shape where the dorsal and ventral portions are higher than the central portions. This is to ensure that the attachment flange can adhere properly to the Cena Ani and Perineum of the user when collecting faecal matter from the anus. In the case where the attachment member should be designed to be attached to a stoma, the attachment flange could be arranged in a more planar manner. However, in the case where the attachment flange is flexible and stretchable enough, then the same attachment flange could be used to connect to both stoma and anus as it will be able to adapt to the different geometries of the different applications. In some embodiments, the attachment flange could have a cut-out or a portion with a reduced thickness to make room for a user's body parts or to allow for more stretchability around a user's body parts. This could for example be the portion near the perineum. In particular, in some embodiments, the attachment flange could be provided with a cut-out, which is arranged to give room for a scrotum of a male user or a vagina of a female user.

In some embodiments for female users, the attachment flange could be provided with a second opening arranged to be in fluid communication with the urinary opening of the female user, said attachment flange comprising an adhesive layer portion arranged between the first and second openings. In some such embodiments for females users, the attachment member could comprise a second tubular portion having a first end and a second end, said first end being in fluid communication with the second opening in the attachment flange and said second tubular portion providing fluid communication between the second opening in the attachment flange and the second end of the second tubular portion. It should be noted that as mentioned above, features mentioned in regards to one invention can be applied to another invention as well. For example, the shape of the attachment flange described here, could also be relevant for the shape of the attachment flange of the first, third or fourth inventions as well.

In order to apply the attachment member of the body waste collecting system to a user, it may be beneficial for the body waste collecting system to comprise an applicator to help apply at least a portion of the attachment member to the user. It may be beneficial that such an applicator somehow supports at least a portion of the attachment flange of the attachment member during application and/or storage. Details of such applicators are not provided in this specification, but different possibilities are disclosed in applicants co-pending applications WO2018/050856 and WO2019/179586 which are incorporated by reference.

While a number of specific examples of materials suitable for providing this form of behaviour are provided herein, other forms and types of material should be able to be provided by the person skilled in the art of materials. Hence, the scope of protection should not be limited to the specific compositions of materials mentioned herein.

In some embodiments, the flange member of the first portion of the attachment flange is made of an elastomeric material such as TPE. In one embodiment, the flange member of the first portion of the attachment flange is made of a silicone material such as Liquid Silicone Rubber. In one embodiment, the flange member of the first portion of the attachment flange is a made of a material with a hardness of shore A90 or less, of shore A60 or less, of shore A45 or less or of shore A5 to shore A40. In one embodiment, the flange member of the first portion of the attachment flange has an average material thickness of less than 1 mm excluding the adhesive. In some embodiments, the flange member can be thicker than 0.1 mm, 0.2 mm, or 0.3 mm. In some embodiments, the flange member can be between 0.3 and 0.9 mm thick.

Some users have increased skin perspiration (moist evaporation from the surface of the skin), which may be due to any number of different variables or factors. In one example, a user might suffer from fever or a user might have recently damaged some skin. In both these examples, the user will experience increased perspiration. In general, most users will experience some form of sweat or perspiration at certain times. Moisture is known to reduce the adhesive power of systems adhered to the skin.

Some prior art adhesives (such as Hydrocolloid adhesives) deal with skin perspiration via absorption as they to a large extent store condensed moisture in the adhesive matrix itself. As the moisture is absorbed, the adhesive will slowly loose its adhesive power. Other (non-absorbent adhesive systems) will not be able to absorb any moisture. When the skin perspiration increases, moisture is built-up between the adhesive and the skin, resulting in reduced or lost adhesive power.

When attaching an attachment flange comprising an adhesive to the skin of a user, it may therefore be desirable to provide a system which allows the skin perspire to escape the system. This ensures a more robust connection. For an attachment member of a body waste collecting system, one could allow the attachment flange to have a certain permeability to water vapour. Perspired moisture would then migrate through the flange and into the atmosphere, thereby maintaining the strength of the adhesive joint between the skin and the attachment member. Removal of moisture will also keep the skin underneath the attachment member healthier than if the skin was more occluded. Increased permeability to water vapour can be provided in different ways. Some different non-limiting options are provided in applicant's co-pending application WO2019/179586.

In one embodiment, the average width of the attachment flange is between 12 and 30 mm, between 14 and 27 mm or between 16 and 24 mm. In another embodiment, the minimum width of the attachment flange is greater than 8 mm, greater than 10 mm or greater than 15 mm.

It should be noted that the embodiments shown in the figures show an attachment flange with a roughly circular outer periphery. However within the scope of the current invention, the outer periphery of the attachment flange could take other forms and does not have to be circular.

Some examples of the second invention are provided below. These examples could form the basis of claims in a divisional application.

Example 1. A system for collecting body waste from a waste expulsion orifice of a user, for example an anus or a stoma, said system comprising:
a. an attachment member arranged to be attached to skin surrounding said orifice, said attachment member comprising
   i. an attachment flange which is arranged to be attached to said skin such that a fluid tight seal is established between the attachment flange and said skin entirely around said orifice when the attachment member is attached to the user, said attachment flange comprising a skin friendly adhesive layer with a skin facing surface,
   ii. an opening in the attachment flange which is in fluid communication with said orifice when the attachment member is attached to the user,
   iii. a tubular portion having a first end and a second end, said first end being connected to the attachment flange via a ring formed connection surrounding the opening in the attachment flange and said second end being arranged spaced away from the attachment flange and spaced away from the user when the attachment member is attached to the user, said tubular portion providing a fluid connection between the opening in the attachment flange and the second end of the tubular portion,
characterized in that the attachment flange comprises a first portion extending between an inner periphery of the first portion and an outer periphery of the first portion arranged spaced away from the inner periphery of the first portion in a direction away from the opening in the attachment flange and a second portion extending between the outer periphery of the first portion and an outer periphery of the second portion which is further away from the opening in the attachment flange than the outer periphery of the first portion, said first portion having an elongation per unit force applied along the attachment flange and said second portion having a different elongation per unit force applied along the attachment flange which is higher than the elongation per unit force of the first portion. In some embodiments, the elongation per unit force of the second portion is at least 200%/Newton for a 15 mm wide strip of material of the second portion. In some embodiments, the elongation per unit force of the central portion is at least 200%/Newton or at least 250%/Newton for a 15 mm wide strip of material of the central portion. In some embodiments, the elongation per unit force of the second portion could be at least 2, 4, 6 or 8 times greater than the elongation per unit force of the first portion.

Example 2. A system according to example 1, characterized in that the first portion is at least 2 mm wide and in that the second portion is at least 5 mm wide.

Example 3. A system according to example 1 or 2, characterized in that said first portion comprises a flange member which is connected to the tubular portion at the ring formed connection and an adhesive layer arranged on the skin facing surface of the flange member and in that the second portion comprises an adhesive layer with a skin facing surface and a backing layer on the surface of the adhesive layer facing away from the skin, said backing layer being less adhesive than the adhesive layer.

Example 4. A system according to example 3, characterized in that the adhesive layer of the first portion and the adhesive layer of the second portion have a shore harness of Shore A: 10 or less.

Example 5. A system according to example 3 or 4, characterized in that the backing layer has an elongation per unit force along the attachment flange which is larger than the elongation per unit force along the attachment flange of the flange member.

Example 6. A system according to any one of examples 1 to 5, characterized in that the second portion has a shore hardness of Shore A: 10 or less.

Example 7. A system according to any one of examples 1 to 6, characterized in that the attachment flange further comprises a central portion, said central portion extending from an inner periphery of the first portion towards the opening in the attachment flange, said first portion having an elongation per unit force applied along the attachment flange and said central portion having a different elongation per unit force applied along the attachment flange which is higher than the elongation per unit force of the first portion. In some embodiments, the elongation per unit force of the central portion is at least 150%/Newton for a 15 mm wide strip of material of the central portion. In some embodiments, the elongation per unit force of the central portion is at least 200%/Newton or at least 250%/Newton for a 15 mm wide strip of material of the central portion.

Example 8. A system according to example 7, characterized in that the width of the central portion is at least 2 mm.

Example 9. A system according to example 7 or 8, characterized in that the central portion has a shore hardness of Shore A: 10 or less.

Example 10. A system according to any one of examples 1 to 9, characterized in that the attachment flange and the tubular portion are made of essentially non-water-absorbing materials.

As mentioned above, this specification also discloses a third invention. The third invention is described in the detailed description as well as via examples provided below. These examples could form the basis of claims in a divisional application.

Example 11. A system for collecting body waste from a waste expulsion orifice of a user, for example an anus or a stoma, said system comprising:
a. an attachment member arranged to be attached to skin surrounding said orifice, said attachment member comprising
   i. an attachment flange which is arranged to be attached to said skin such that a fluid tight seal is established between the attachment flange and said skin entirely around said orifice when the attachment member is attached to the user, said attachment flange comprising a skin friendly adhesive layer with a skin facing surface,
   ii. an opening in the attachment flange which is in fluid communication with said orifice when the attachment member is attached to the user,
   iii. a tubular portion having a first end and a second end, said first end being connected to the attachment flange via a ring formed connection surrounding the opening in the attachment flange and said second end being arranged spaced away from the attachment flange and spaced away from the user when the attachment member is attached to the user, said tubular portion providing a fluid connection between the opening in the attachment flange and the second end of the tubular portion,
characterized in that the attachment flange comprises a first portion extending between an inner periphery and an outer periphery arranged spaced away from the inner periphery in a direction away from the opening in the attachment flange and a central portion extending from the inner periphery of the first portion towards the opening in the attachment flange, said first portion having an elongation per unit force applied along the attachment flange and said central portion having a different elongation per unit force applied along the attachment flange which is higher than the elongation per unit force of the first portion. In some embodiments, the elongation per unit force along the attachment flange of the central portion is at least 2, 4, 6 or 8 times greater than the elongation per unit force of the first portion. In some embodiments, the inner periphery of the first portion is arranged at an inner periphery of the ring formed connection. In some embodiments the outer periphery of the first portion is arranged at an outer periphery of the ring formed connection. In some embodiments, the elongation per unit force of the central portion is at least 150%/Newton for a 15 mm wide strip of material of the central portion. In some embodiments, the elongation per unit force of the central portion is at least 200%/Newton or at least 250%/Newton for a 15 mm wide strip of material of the central portion.

Example 12. A system according to example 11, characterized in that the central portion has a shore hardness of Shore A: 10 or less, Shore A: 5 or less, Shore A: 2 or less, Shore 00: 30 or less, Shore 00: 20 or less or Shore 000: 35 or less. In some embodiments, said first portion has a first shore hardness and said central portion has a second shore hardness which is softer than the first shore hardness.

Example 13. A system according to example 11 or 12, characterized in that the width of the central portion is greater than 2 mm, 3 mm, 4 mm or 5 mm and in that the width of the first portion is greater than 2 mm, 3 mm, 4 mm or 5 mm.

Example 14. A system according to any one of examples 11 to 13, characterized in that said first portion comprises a flange member which is connected to the tubular portion at the ring formed connection and an adhesive layer arranged on the skin facing surface of the flange member and in that the central portion comprises an adhesive layer having an adhesive surface facing the skin.

Example 15. A system according to example 14, characterized in that the adhesive layer of the first portion and the adhesive layer of the central portion form a single connected adhesive layer. In some embodiments, the thickness of the adhesive layer could be greater than 1 mm, 2 mm or 3 mm.

Example 16. A system according to any one of examples 11 to 15, characterized in that the attachment member is made of essentially non-water-absorbing materials. In this way, it is possible to clean and re-use the attachment member.

Example 17. A system according to any one of examples 11 to 16, characterized in that the attachment flange further comprises a second portion extending between the outer periphery of the first portion and an outer periphery of the second portion which is further away from the opening in the attachment flange than the outer periphery of the first portion, said first portion having an elongation per unit force applied along the attachment flange and said second portion having a different elongation per unit force applied along the attachment flange which is higher than the first elongation per unit force. In some embodiments, the elongation per unit force of the second portion could be at least 2, 4, 6 or 8 times greater than the elongation per unit force of the first portion.

Example 18. A system according to example 17, characterized in that the second portion is at least 5 mm wide. In some embodiments, the second portion could be at least 10 mm, at least 15 mm or at least 20 mm wide.

Example 19. A system according to example 14 and example 17 or 18, characterized in that the second portion comprises an adhesive layer with an adhesive surface facing the skin and a backing layer on the surface of the adhesive layer facing away from the skin, said backing layer having an exposed surface which is less adhesive than the exposed surface of the adhesive layer.

Example 20. A system according to example 19, characterized in that the adhesive layer of the first portion, the adhesive of the central portion and the adhesive of the second portion form a single connected adhesive layer. In some embodiments, the connected adhesive layer could have a shore harness of Shore A: 10 or less, Shore A: 5 or less, Shore A: 2 or less, Shore 00: 30 or less, Shore 00: 20 or less or Shore 000: 35 or less.

As also mentioned above, the current specification discloses a fourth invention related to a hybrid system. Examples of such a hybrid system are provided below. These examples could form the basis of a divisional application in the future.

Example 21. A system for collecting body waste from a waste expulsion orifice of a user, for example an anus or a stoma, said system comprising:
a. an attachment member arranged to be attached to skin surrounding said orifice, said attachment member comprising:
   i. an attachment flange which is arranged to be attached to said skin such that a fluid tight seal is established between the attachment flange and said skin entirely around said orifice when the attachment member is attached to the user, said attachment flange comprising a skin friendly adhesive layer with a skin facing surface,
   ii. an opening in the attachment flange which is in fluid communication with said orifice when the attachment member is attached to the user,
   iii. a tubular portion having a first end and a second end, said first end being connected to the attachment flange via a ring formed connection surrounding the opening in the attachment flange and said second end being arranged spaced away from the attachment flange and spaced away from the user when the attachment member is attached to the user, said tubular portion providing a fluid connection between the opening in the attachment flange and the second end of the tubular portion, and
   iv. a bag engaging portion arranged at the second end of the tubular portion, said bag engaging portion having an opening and a rim portion surrounding the opening,
b. a first collection bag, said collection bag comprising
   i. a reservoir for collecting and storing faecal matter from the user, and
   ii. an attachment member engaging portion for detachably attaching the collection bag to the bag engaging portion of the attachment member, and
c. an elongated tubular conduit having a length of more than 30 cm, said elongated conduit having a first end which is arranged with an attachment member engaging portion for detachably attaching the first end of the elongated tubular conduit to the bag engaging portion of the attachment member to establish fluid communication between the opening in the attachment member and the elongated conduit and a second end connected to a collection bag comprising a reservoir for collecting and storing faecal matter from the user.

Example 22. A method of using a system according to example 21, characterized in that in a first step the attachment member is applied to a user, in a second step, the first collection bag is attached to the bag engaging portion of the attachment member, in a third step the first collection bag is detached from the bag engaging portion of the attachment member, and in a fourth step, the first of the elongated tubular conduit is attached to the bag engaging portion of the attachment member.

In this way, a system is provided which can be used both with a collection bag connected directly to the attachment member, and with a collection bag attached to the attachment member via a conduit. All prior art systems are either bag solutions where a bag is attached to a user, or conduit based system where a bag is spaced away from a user. However, in certain cases, for example during the transfer of a patient from one location to another, it is advantageous to be able to switch between a local bag and a remote bag without having to replace the attachment member on the user.

It will be appreciated that any combination of features and elements of the above described aspects, inventions, examples and/or embodiments may be combined in any suitable manner.

It should be emphasized that the term “comprises/comprising/comprised of” when used in this specification is taken to specify the presence of stated features, integers, steps or components but does not preclude the presence or addition of one or more other features, integers, steps, components or groups thereof.

### BRIEF DESCRIPTION OF THE DRAWINGS

In the following, the invention will be described in greater detail with reference to embodiments shown by the enclosed figures. It should be emphasized that the embodiments shown are used for example purposes only and should not be used to limit the scope of the invention.

FIG. 1 schematically shows a system according to one embodiment of the second invention.

FIG. 2 shows a cross section through a first embodiment of an attachment member according to the second invention, said cross section taken on the sagittal plane of a user when the attachment member is attached to a user.

FIG. 3 shows a cross section through the attachment member of FIG. 2, said cross section taken on the coronal plane of a user when the attachment member is attached to a user.

FIG. 4 shows a schematic perspective view of a second embodiment of an attachment member according to the second invention.

FIG. 5 shows a cross section of the attachment member of FIG. 4, taken on the sagittal plane of a user when the attachment member is attached to a user.

FIG. 6 shows a detailed view of the attachment member of FIG. 4, according to the circle marked with VI in FIG. 5.

FIG. 7 schematically shows a cross section of a third embodiment of an attachment member according to the second and third inventions, the cross section taken on the sagittal plane of a user when the attachment member is attached to a user.

FIG. 8 shows a schematic perspective view of a fourth embodiment of an attachment member according to the second invention.

FIG. 9 shows a cross section of a fifth embodiment of an attachment member according to the second and third inventions, taken on the sagittal plane of a user when the attachment member is attached to a user.

FIG. 10 schematically shows a cross section of a fourth embodiment of an attachment member according to the second and third inventions attached to a stoma of a user.

FIG. 11 schematically shows an embodiment of a system according to the first invention.

FIG. 12 shows a second view of the attachment member of the system of FIG. 11 showing some descriptive dimensions.

FIG. 13 shows a schematic cross section view of another embodiment of an attachment member of a system according to the first invention, prior to attaching the bag to the attachment flange and prior to attaching the attachment flange to a user.

FIG. 14 shows a schematic cross section view of the embodiment of FIG. 13, but where the bag has been attached to the bag engaging portion and the attachment flange has been attached to a user.

FIG. 15 shows a schematic cross section view of another embodiment of an attachment member of a system according to the first invention, prior to attaching the bag to the attachment flange and prior to attaching the attachment flange to a user.

FIG. 16 shows a second view of the attachment member of the system of FIG. 15 showing some descriptive dimensions.

FIG. 17 shows a schematic cross section view of the embodiment of FIG. 15, but where the bag has been attached to the bag engaging portion flange and the attachment flange has been attached to a user.

FIG. 18 schematically shows a cross section through an embodiment of an attachment member according to the first invention, said cross section taken on a sagittal plane of a user when the attachment member is attached to the user.

FIG. 19 schematically shows a cross section through the attachment member of FIG. 18, said cross section taken on a plane which is perpendicular to the sagittal plane when the attachment member is attached to the user and parallel to a central axis passing through the tubular portion.

FIG. 20 schematically shows a bottom view of the attachment member of FIGS. 18 and 19.

FIG. 21 schematically shows a cross section through an embodiment of an attachment member according to the first invention, said cross section taken on a sagittal plane of a user when the attachment member is attached to the user.

FIG. 22 schematically shows a cross section through the attachment member of FIG. 21, said cross section taken on a plane which is perpendicular to the sagittal plane when the attachment member is attached to the user and parallel to a central axis passing through the tubular portion.

FIG. 23 schematically shows a test sample for testing the stretchability of a material.

### DETAILED DESCRIPTION OF THE EMBODIMENTS

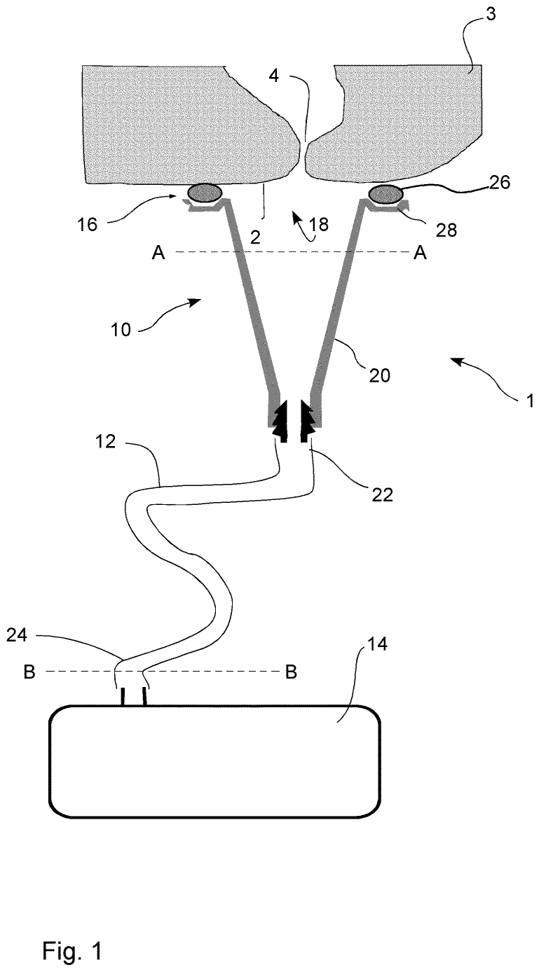

FIG. 1 discloses a very schematic illustration showing the main components of the body waste collecting system in the form of a perianal faecal collecting system as an example of a system according to the second invention when attached to a user.

The system 1 is arranged to be attached to the perianal skin 2 of a user 3 around the anus 4 and collect faecal waste exiting the anus of the user. The system 1 in general comprises an attachment member 10, a tubular conduit 12 and a collection bag 14. The attachment member is arranged to establish a fluid tight connection between the system and the user. The conduit is arranged to establish fluid communication between the attachment member and the collection bag. The collection bag is arranged to collect the faecal output. This type of system is described in more detail in applicant's co-pending applications WO2018/050856 and WO2019/179586 which are included by reference in their entirety.

The attachment member 10 comprises an attachment flange 16 which is attached to the perianal skin of the user. The attachment member 10 of the current embodiment also comprises an opening 18 and a tubular portion 20. The conduit 12 has two ends, a first end 22 in fluid communication with the attachment member and a second end 24 in fluid communication with the collection bag.

In the current schematic embodiment, there are three separate elements, an attachment member 10, a conduit 12 and a collection bag 14. However, within the scope of the invention, these elements could be integrated together in different combinations. For example, an embodiment could be made which comprises a single integrated element comprising an attachment member, a conduit and a collection bag. In another embodiment, the conduit and attachment member could be integrated into a single element and the collection bag could be connected to the free end of the conduit, or the conduit and the collection bag could be integrated into a single element and the free end of the conduit could be connected to the attachment member.

Likewise, it should be mentioned that the tubular conduit and the tubular portion of the attachment member fulfil similar functions. For the sake of the current specification and for the understanding of the claims, the attachment member should be understood as the part of the system which establishes a fluid tight communication with the waste expulsion orifice of a user, the conduit should be understood as that part of the system which establishes fluid communication between the attachment member and the collection bag, and the collection bag is the part of the system which collects the faecal or urinary waste from the user. In certain embodiments, it could be imagined that the collection bag was directly connected to the attachment member, thereby having the collection bag close to the user. It could also be imagined that the tubular portion of the attachment member was very short, effectively 1 mm or less in length just to provide a connection between the attachment flange and a bag or conduit. In this way, the collection bag could be connected directly to the attachment flange, just via some form of connection portion. Examples of this situation are discussed below.

In the embodiment shown in FIG. 1, it can therefore be understood that the portion of the system which is above the line AA is considered the attachment member, the portion which lies under the line BB is considered the collection bag and the portion which lies between the lines AA and BB is considered the conduit. Using this understanding, in some embodiments the conduit should be at least 30 cm long. In some embodiments, the conduit should be less than 8 cm in diameter. In some embodiments, the conduit and/or the tubular portion of the attachment member could be greater than 1 cm, 2 cm, 3 cm or 4 cm in diameter.

In the current embodiment shown in FIG. 1, the attachment flange 16 is attached to the perianal skin via an adhesive 26. It should be noted that in the applicant's previously filed applications, it has been described that an adhesive was applied to or on the attachment flange. However, in the current specification, the adhesive 26 is considered to be a part of the attachment flange 16, since it has been found that the adhesive is such an integral and important component of the functioning of the attachment flange that it is difficult to separate them into two separate components. As such, in the figure, the attachment flange 16 comprises a backing layer 28 or flange member 28 and the adhesive is applied on the backing layer. As will be discussed later on, the different properties and dimensions of the adhesive and the backing layer can be chosen to provide attachment flanges with different properties.

As has been mentioned earlier on, many suitable forms of adhesive are available. It can also be noted that adhesive and mechanical systems which attach the system to the user via straps, belts, etc could also be combined if desired.

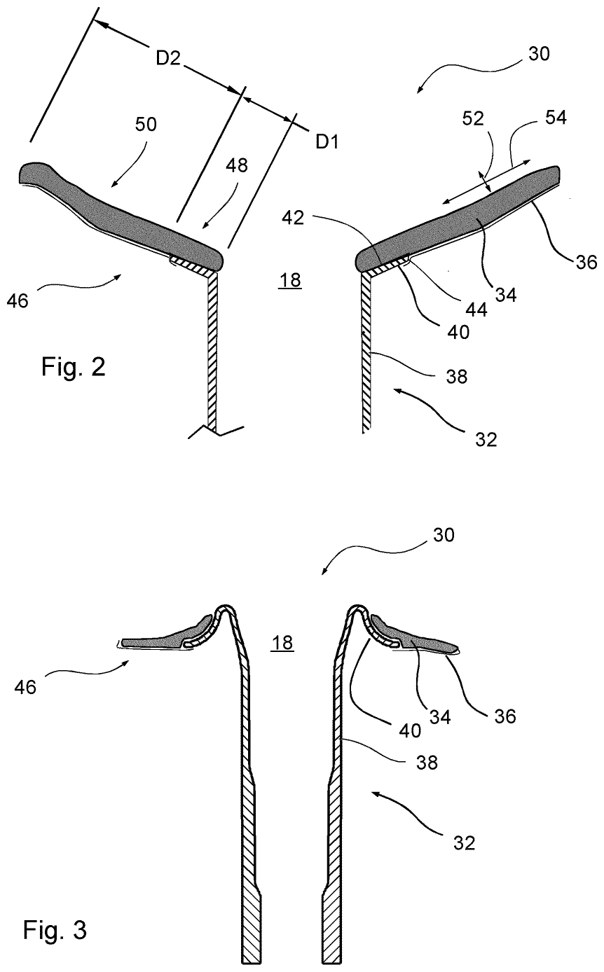

In FIGS. 2 and 3 a more complete, but still schematic, illustration of an attachment member 30 according to the second invention is shown via two cross section views. The first cross section view is taken along a sagittal plane and the second cross section view is taken along the coronal plane with respect to a user if the attachment member had been attached to a user. Note that by coronal plane is meant a plane which is perpendicular to the sagittal plane and arranged such that it passes through the central portion of the attachment member. It could be slightly different from the more medically correct definition of the coronal plane of a user. In this embodiment, the attachment member comprises a moulded silicon portion 32, an adhesive layer 34 and a thin backing layer 36. The silicon portion comprises a tubular portion 38 and a flange member 40 attached at one end of the tubular portion and extending outwardly from the tubular portion. The flange member 40 has a skin facing side 42 on which the adhesive layer 34 is arranged. However, as can be seen, the adhesive extends away from the center of the attachment member further out than the outer periphery 44 of the flange member 40. The thin backing layer 36 is arranged on the surface of the adhesive which extends past the outer periphery of the flange member 40 and which faces away from the skin. The thin backing layer is a non-adhesive material so that the surface of the adhesive facing away from the skin does not stick to undesired things.

As such, the attachment member comprises an attachment flange 46 which is comprised partly of the flange member of the silicon portion, an adhesive layer and a thin backing layer. The attachment flange also has two clearly defined portions, a first “inner” portion 48 comprising the flange member of the silicon portion and the adhesive layer covering the flange member and a second “outer” portion 50 comprising the adhesive layer extending past the flange portion and the thin backing layer. The width of the first portion is provided by the dimension D1 in the figure and the width of the second portion is provided by the dimension D2 in the figure. In one embodiment, the dimension D1 is 5 mm and the dimension D2 is 17 mm. However, many other dimensions could also be imagined.

Since the tubular portion is connected directly to the flange member, any forces applied to the tubular portion 38 are transferred to the flange member of the silicon portion, and thereby on to the adhesive layer of the attachment flange. However, the forces are not directly transferred to the outer periphery of the attachment flange since the flange member of the silicon portion does not extend out to the outer periphery of the attachment flange. In this way, the outer periphery of the attachment flange will not be pulled away from the skin and the flange will stay better connected to the skin for longer periods of time.

When describing the properties of the attachment flange, one property used is the amount of elongation of the flange when a force is applied to it. Depending on the direction of the force, the components of the flange, and the material properties and dimensions of the components of the flange, the elongation will be different. For example a thick layer of a soft material or a very thin layer of a harder material, might have the same elongation for the same force. When a force is applied perpendicular to the flange as shown by the arrow 52 in FIG. 2, the different layers of the flange will act in series and the layer with the softest material will stretch more than the other layers. In contrast when a force is applied along the flange as shown by the arrow 54 in FIG. 2, the different layers will act in parallel and all the layers will stretch essentially the same amount. Hence, the properties when applying force along the flange will be different than when applying force perpendicular to the flange.

In this embodiment, the attachment flange has a width of around 22 mm all the way around the opening of the attachment member. Furthermore, the skin contacting surfaces of the attachment flange at the dorsal and ventral portions of the attachment flange are shown to form an angle to each other of around 130 degrees in the figures. This angle allows the dorsal and ventral portions to better contact the Crena Ani and Perineum portions of the user more effectively. This angle could be formed either directly in the moulded silicon portion, or it could be provided in that the silicon portion is very flexible and the attachment flange can easily bend to adapt to the specific shape of the user. In the current invention, where the second portion of the attachment flange is very flexible and stretchable, the attachment flange will adapt very easily to the shape of the user.

FIG. 3 shows the attachment member of FIG. 2 in a cross section view taken along a coronal plane of a user when the attachment member is attached to a user.

In this case, the portions of the attachment flange, extend downwards away from the opening of the attachment member in contrast to the dorsal and ventral portions which extend upwardly. This illustrates very well, the complex geometry which the attachment flange needs to adapt to in the perianal situation. This is different in stoma solutions where the attachment flange typically attaches to the outer surface of the belly of a user which has a much more well defined and flat or just slightly curved surface. In the perianal situation, parts of the flange need to bend upwards and parts bend downwardly. Similarly, during motion of the user, the users legs and buttocks and other parts can all move in different directions, again in contrast to a stoma solution where the belly surface is pretty much constant.

It should be noted that in the figures above and in the further embodiments shown in the following figures, the flange member extends outwardly from the tubular portion. In this way, the first portion of the attachment flange is arranged “outside” the tubular portion. However, it would also be possible for the flange member to extend inwardly from the tubular portion. In this way, the first portion of the attachment flange would be “inside” the tubular portion. In such an embodiment, a larger diameter of the tubular portion might be required.

It should also be noted that in these figures and in the later figures, spaces are often shown between the different components of the device. For example a small gap is shown between the backing layer 36 and the adhesive layer 34 of the second portion 50 of the attachment flange. However, this is to illustrate the different components of the device more clearly. In a real embodiments, there would be no air gap and the elements would be joined directly together. It is maintained that the person skilled in the art will understand this based on the teachings of this specification and his/her common general knowledge.

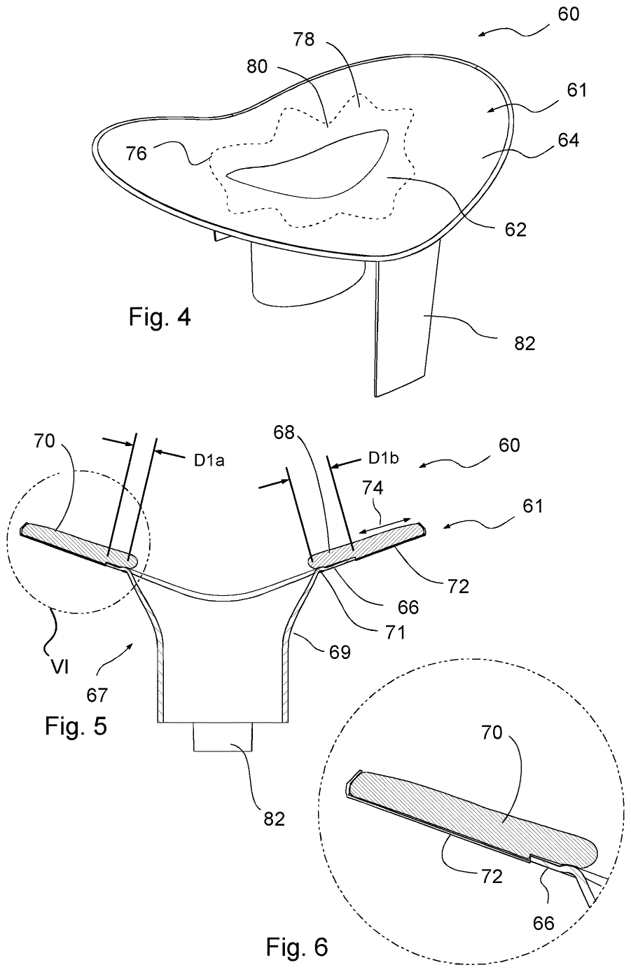

FIGS. 4-6 show some different views of a second embodiment 60 of an attachment member according to the first invention. As with the previous embodiment, the attachment flange 61 comprises a first portion 62 and a second portion 64 arranged outside the first portion. The first portion comprises a flange member 66 and an adhesive layer 68 arranged on the skin facing side of the flange member 66. The second portion comprises an adhesive layer 70 and a thin backing layer 72. As before, the thin backing layer mainly serves to cover the adhesive layer to make sure that the adhesive is not active on the surface facing away from the skin. However, it should be noted that in some embodiments, the thin backing layer can also be used to make production of the device easier. For example, a thin backing layer can be provided as a foil layer attached to the flange member and then the adhesive layer can be applied on top of the thin backing layer and the flange member. In this case, the thin backing layer can provide support for the adhesive layer during the manufacturing of the attachment flange.

However, in general, the thin backing layer does not contribute in any significant way to the stretching force of the adhesive layer parallel to the plane of the attachment flange. In this embodiment, the thin backing layer is provided by painting or spraying a thin film onto the surface of the adhesive layer. As shown by the arrow 74, the second portion of the attachment flange is therefore very stretchable and this portion can displace much more easily when compared to the flange member 66.

In this case, the flange member 66 is also provided as one part of a moulded silicon portion 67 which also comprises a tubular portion 69. The tubular portion is connected to the attachment flange via the ring formed connection 71. In this case, the flange member 66 of the silicon portion has different widths defined by an undulating outer periphery 76. In this way, certain sections 78 will have greater area of attachment to the adhesive than other sections 80. This is illustrated by the different widths of the flange portion shown by the dimensions D1a and D1b.

FIG. 4 also shows straps 82 arranged on either side of the attachment flange. The straps are made from a flexible material, for example a rubber sheet like material. The straps are used to pull back the attachment flange during attachment to the user. The helper or the user pulls the straps backwards, thereby causing the sides of the attachment flange to lay flat against the sides of the tubular portion. The attachment member can then be more easily inserted against the perineum and Crena Ani. Once in place, the sides of the flange can be released and pushed outwards to attach to the sides of the perianal skin. The straps also support the removal of the product after use. In this embodiment, the straps are attached to the outer periphery of the attachment flange and are not directly connected to the flange member of the silicon portion.

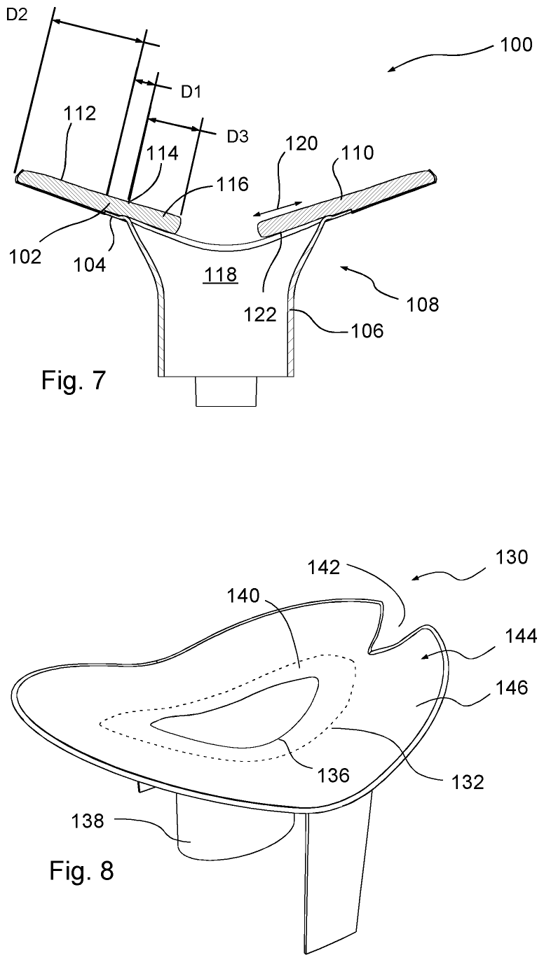

FIG. 7 shows a third embodiment 100 of an attachment member of the second invention, but also shows a feature of the third invention. In this case, the attachment flange has a first portion 102 which comprises a flange member 104 connected to the tubular portion 106 of a moulded silicon portion 108. An adhesive layer 110 is formed on the flange member. As before the adhesive layer extends out past the end of the flange member to form a second portion 112 of the attachment flange. In addition, the adhesive layer extends inwardly past the inward edge 114 of the first portion to form a third or “central” portion 116. The central portion and the second portion are both very soft and stretchable. In this way, the central portion can also adapt itself very well to changing geometry. In for example a perianal situation, the inner diameter of the opening 118 defined by the central portion will adapt itself to the opening and closing of the anus. This is shown by the arrow 120. The surface 122 of the central portion which faces away from the skin could be covered by some form of backing layer as well.

FIG. 8 shows another embodiment 130 of an attachment member which is very similar to the embodiment of FIG. 4, however, instead of having an undulating edge of the first portion, in this embodiment, the edge 132 of the first portion 134 follows the outline of the ring formed connection 136 between the tubular portion 138 and the flange member 140 (underneath the adhesive layer). However, in this embodiment, a cutout 142 is provided in the attachment flange 144 at the side of the attachment flange which is closest to the perineum when the attachment flange is attached to the user. In this way, there can be room for the scrotum or vagina of the user depending on whether the user is male or female. Since the second portion (outer portion) 146 of the attachment flange is very soft, the attachment flange can be easily adapted in shape to fit around the scrotum or vagina of the user. In certain cases, not shown, the attachment flange could comprise a second opening, for example being in fluid communication with the vagina of a female user. A second tubular portion could be provided in fluid communication with the second opening.

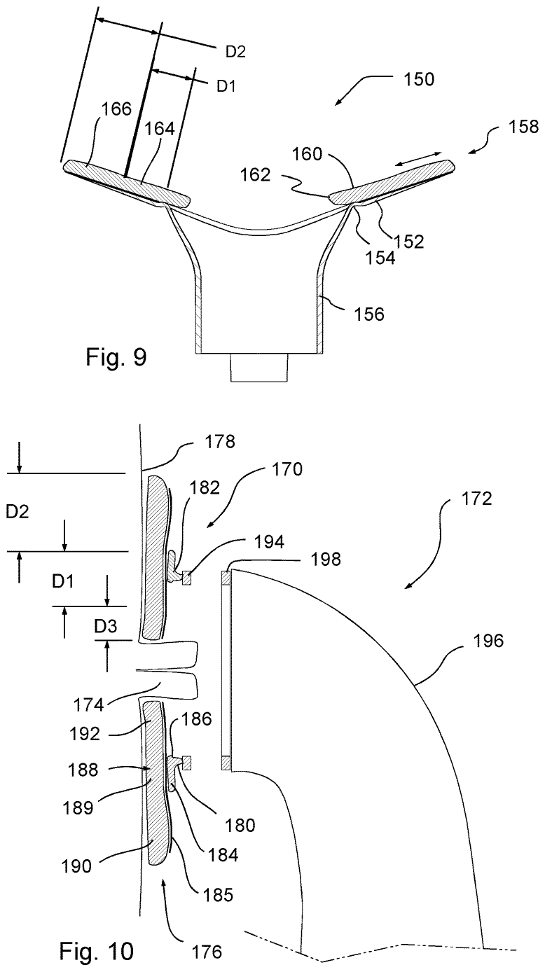

FIG. 9 shows another embodiment 150 of an attachment member. For the most part the features are the same as the embodiment of FIG. 7 and as such, the details will not be described. However, the difference is in the formation of the flange member and the backing layer. In this case, instead of having a flange member and then a separate thin backing layer, a flange member 152 is provided which extends from the ring formed connection 154 between the flange member and the tubular portion 156 all the way to the outside of the attachment flange 158. However, the flange member is provided with a decreasing thickness as it extends outwardly. In this way, a “tapering flange member” is provided where the outer portion of the flange member will be much more stretchy than the inner portion of the flange member. An adhesive layer 160 is formed on the tapering flange member. The adhesive layer also extends inwardly past the inner edge of the ring formed connection 154 to form a soft central portion 162.

In this case, there is no sharp distinction between the first portion and the second portion, however, a first portion 164 identified by the dimension D1 and a second portion 166 identified by the dimension D2 are shown in the figure. Due to the difference in thickness of the flange member in the two portions, the elongation of the two portions when exposed to the same force will be very different and the outer portion will stretch much more and the inner portion will stretch much less.

FIG. 10 shows an embodiment 170 of an attachment member and a collection bag 172 which is detachably connected to the attachment member. This attachment member is suitable for attaching to a stoma 174. In general, however, the attachment member is very similar in principle to the attachment member of FIG. 7. The attachment member comprises an attachment flange 176 which attaches to the peristomal skin 178 around the stoma and a tubular portion 180 connected to the attachment flange at the ring formed connection 182. The attachment flange also comprises a flange member 184 connected to a first end 186 of the tubular portion 180.

On the skin facing side of the flange member, a thin backing layer 185 in the form of a silicone film is provided and on a skin facing side of the thin backing layer, an adhesive layer 188 is applied. The thin backing layer extends out past the outer periphery of the flange member as well as inwardly past the inner periphery of the first portion. In this way, the adhesive layer and the thin backing layer arranged directly connected to the flange member form a first portion 189 of the attachment flange. The adhesive layer and the thin backing layer extending outwardly past the outer end of the flange member form an outer or “second” portion 190. The adhesive layer and the thin backing layer extending inwardly past the inward end of the flange member form a central or “third” portion 192. The function of the first, second and third portions are the same as described above with respect to the other embodiments.

On the second end of the tubular portion is arranged a bag engaging portion 194. The bag engaging portion could take many forms as is known in the art of ostomy pouches. It should be noted that in this embodiment, the tubular portion is very short. It could even be imagined that the bag engaging portion is attached directly to the attachment flange via a ring formed connection. In this case, the tubular portion would be essentially removed. However for the sake of this specification, even in the case where the bag engaging portion is connected directly to the attachment flange, it can still be considered that there is a tubular portion connecting the attachment flange and the bag engaging portion, it is just very short.

The detachable collection bag 172 comprises a flexible foil bag 196 and a cooperating attachment member engaging portion 198 which can be detachably connected to the bag engaging portion of the attachment member. In another embodiment (not shown), the collection bag could be connected directly to the tubular portion on the attachment member. The advantage of this embodiment with respect to the prior art is that the central portion attaches securely to the peristomal skin in a soft way. Any changes in size of the stoma will be easily absorbed by the central portion. Likewise, the outer portion also ensures that the attachment flange has a secure connection to the skin of the user and loads on the ostomy pouch will not cause the outer portion of the flange to detach from the skin of the user.

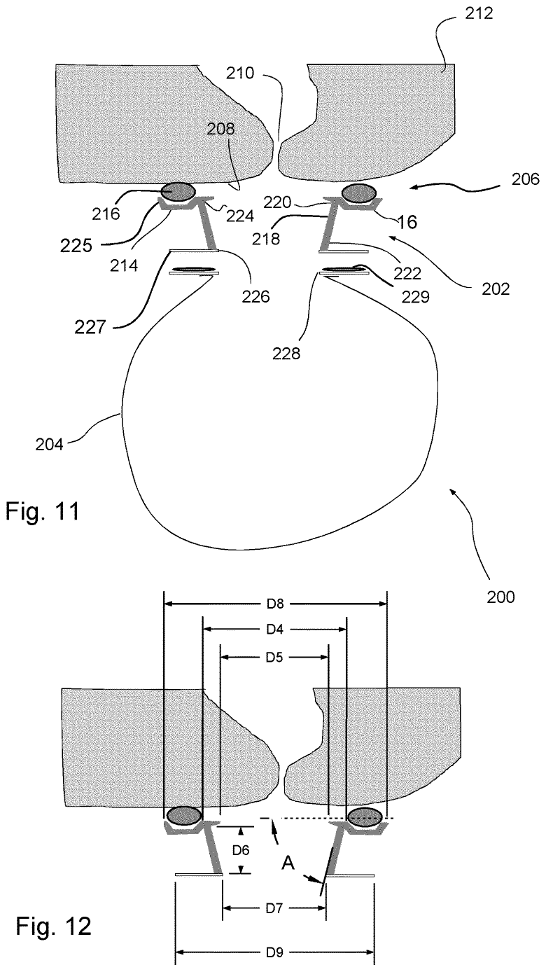

FIG. 11 shows a schematic illustration of a system 200 according to the first invention. The system comprises an attachment member 202 and a collection bag 204 detachably connected to the attachment member. The attachment member comprises an attachment flange 206 which is attached to the perianal skin 208 surrounding the anus 210 of the user 212.

The attachment flange comprises a flange member 214 and an adhesive layer 216 arranged on the skin facing side of the flange member. The attachment member comprises a connection portion 218, which in this embodiment is a tubular portion 218 having a first end 220 and a second end 222. The first end is attached to the attachment flange via a ring formed or annular connection 224. In this embodiment, the tubular portion and flange member are integral parts of a moulded silicon component.

The second end of the tubular portion is provided with a bag engaging portion 226. The bag engaging portion is formed as a flexible flange element which extends outwardly from the tubular portion. The flexible flange element is made from a flexible film like material which can bend and adjust its shape. However, the stretchability of the bag engaging portion can be less than the stretchability of the attachment flange. In this embodiment, one could consider the tubular portion 218 together with the flexible flange element 226 as a bag engaging portion connected to the attachment flange.

The collection bag is likewise provided with an attachment member engaging portion 228. In this embodiment, the attachment member engaging portion 228 of the collection bag is also provided as a flexible flange element, in this embodiment welded to the collection bag. A layer of adhesive 229 is applied on the flexible flange element of the attachment member engaging portion on the user facing surface of the flange. The two engaging members are arranged to engage with each other in a detachable, but fluid tight manner. In addition, since both engaging members are flexible, the connection is also flexible. In this embodiment, the skin facing surface of the flange of the attachment member engaging portion is a flexible surface which is attachable to the flexible surface of the flexible flange of the bag engaging portion of the attachment member.

In this embodiment, the distance D4 between the outer most points on the dorsal and ventral portions of the ring formed connection between the tubular portion and the attachment flange is approximately 40 mm. The distance D4 could also be defined as the distance between the outer most points on the ring formed connection on the sagittal plane of a user when the attachment member is attached to a user. Depending on the size of the user, different solutions could be provided. In general, the inner diameter D5 of the opening in the attachment flange should be chosen to be large enough to accommodate the largest expected faecal matter, while the outer distance of the ring formed connection should be chosen small enough to not extend too far into the perineum or Crena Ani. As such the inner diameter (D5) of the opening in the attachment flange should be less than or equal to D4, and the distance D4 should be chosen such that it is large enough to allow a good connection between the attachment flange and the tubular portion, but in general be as small as possible. The outer distance will typically be between 25 mm and 80 mm. For children an outer distance of 30/35 mm would be appropriate and for larger adults, an outer distance of 50/55 mm could be provided.

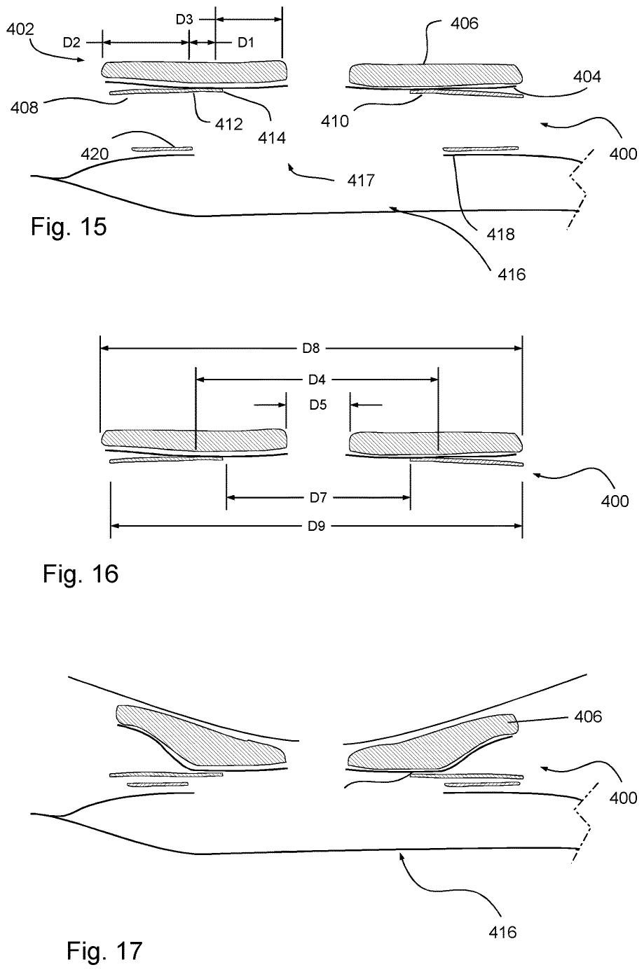

The distance (D6) essentially “perpendicular” to the plane of the attachment flange between the outermost portion of the ring formed connection and the outermost portion of the connection between the bag engaging flange and the tubular portion in the current embodiment is approximately 1 cm in the embodiment of FIG. 11. If the tubular portion is very long, then faecal matter can be “stored” in the tubular portion and it can be difficult to remove when changing the bag. Hence, for the sake of cleaning and convenience, the tubular portion could be less than 3 cm in length. In certain embodiments, as will be illustrated with regards to FIGS. 15 to 17, the distance can be zero where the flange of the bag engaging portion is attached directly to the attachment flange via an annular connection. However, the distance can be adjusted to fit the specific application.

The inner diameter (D7) of the opening in the bag engaging flange should be greater than 20 mm.

The outer diameter D8 of the attachment flange should be greater than the inner diameter of the ring formed connection.

The bag engaging flange should extend outwardly from the connection portion/tubular portion, such that the outer diameter D9 of the bag engaging flange is greater than D7. The dimensions mentioned herein are illustrated in more detail in FIG. 12.

From FIG. 11, it can be seen that the outer periphery 225 of the attachment flange and the outer periphery 227 of the bag engaging flange are not directly connected. As such, the outer periphery of the attachment flange and the outer periphery of the bag engaging flange can move individually. In this way, as the attachment member is applied to the user, the dorsal and ventral portions of the attachment flange can easily bend upwardly and attach securely to the perineum and Crena Ani of the user while the dorsal and ventral portions of the bag engaging flange remain relatively straight. In this way, a flange of the collection bag does not have to deform very much to connect to the bag engaging flange of the attachment member.

It can be imagined that the attachment member is folded in half and then applied to the user between the buttocks around the anus. The dorsal and ventral portions of the attachment flange are bent upwards to securely connect to the perineum and crena ani of the user. The bag engaging flange will also be folded in half due to the press of the buttocks of the user. Then a flange of the collection bag is folded in half, as is known in the art, and the flange applied to the bag engaging flange of the attachment member. Since the flange of the bag and the bag engaging flange of the attachment member are both roughly folded in half, they match in shape and an effective seal is provided. Furthermore, as the user moves, the attachment flange will absorb the motions without the motions being transferred to the bag engaging flange. In this way, the attachment between the user and the attachment member is much more secure and effective when compared to prior art solutions with collection bags attached to the perianal skin of a user.

It can also be noted that by arranging the flexible flange of the bag engaging portion and the flexible flange of the bag to be bendable and arranged between the buttocks of the user during use, the buttocks of the user will squeeze the components together during motion of the user, and thereby increase the effectiveness of the seal between the bag and the attachment member. Furthermore, it has been found that the adhesives used are more effective when warm, and when the components are squeezed together between the buttocks of the user, the adhesive is kept warm and therefore effective. When a bowel movement occurs, the buttocks will part and the flexible flange of the bag engaging portion and the flange of the bag will open and the stool will pass into the collection bag. Once the bowel movement is finished, the buttocks will close, again sandwiching the flexible flange of the bag engaging portion and the flange of the bag between the buttocks.

It should be noted that in the art of stoma pouches, many solutions for detachably connecting pouches to an attachment flange attached to the user around the stoma are known. However in most stoma pouch solutions, the bag engaging portions are connected directly to the attachment flange and are stiff rings which engage with stiff rings on the stoma pouch. This is possible since there is plenty of room for rings and connection mechanisms on the surface of the belly of the user. However, in a perianal embodiment, like the current example, using a stiff ring or other form of standard bag engaging portion attached directly to the attachment flange will not be possible, since the attachment flange needs to adapt to the complex geometry of the user around the perianal skin. This includes the dorsal and ventral portions of the attachment flange bending upwardly to contact the perineum and the Crena Ani and the side portions of the attachment flange bending downwardly to fit between the buttocks of the user. In the embodiment of FIG. 11, the tubular portion can be formed from a flexible thin foil material which can be attached to the attachment flange while still allowing the attachment flange to move and adapt to changing geometries at the perianal skin. Likewise, the tubular portion is allowed to collapse between the buttocks to provide a relatively comfortable fit for the user, even though the tubular portion has a relative large inner diameter. When the user has a bowel movement, the buttocks will move apart as will the sides of the tubular portion and the faecal matter will pass through the tubular portion, through the bag engaging portion and then on into the collection bag.

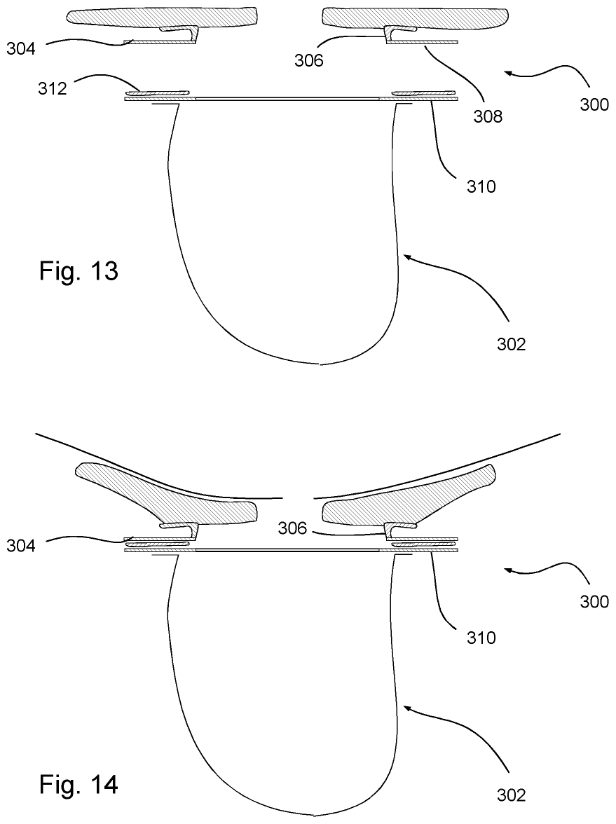

FIGS. 13 and 14 show a schematic cross section view of another embodiment 300 of an attachment member for attachment to the perianal skin for use with a detachable bag 302. In this case, instead of a stiff ring which is held at a distance from the anus, a flexible flange 304 is provided attached to the ring formed connection via a small connection portion 306. It should be clear that the connection portion could be made very small and the flexible flange 304 could be connected almost directly to the attachment flange. The flexible flange provides a surface 308 on which a bag with a similar flexible flange 310 can be attached via an adhesive 312. When the bag flange is attached to the attachment member flange, then the bag and the attachment member are securely connected together. Since the flanges are flexible, the entire system is not uncomfortable for the user to wear as the flexible flanges, the attachment flange and the bag itself are all flexible and will adapt to the shape of the user.

Furthermore, as is shown in FIG. 14, when the attachment flange is attached to the user, the dorsal and ventral portions of the attachment flange can be displaced upwardly to form a firm connection between the perianal skin and the attachment flange. However, the bag engaging portion itself does not need to bend upwardly as does the attachment flange. In contrast, the bag engaging portion can form an essentially straight line at the dorsal and ventral portions and thereby form an effective seal to the bag itself.

FIGS. 15 to 17 show another example 400 of a system for collecting body waste. In this case, the attachment flange 402 comprises a backing layer 404, an adhesive layer 406 and a bag engaging portion 408 in the form of a flat but flexible flange. The inner portion 410 of the flange is attached to the backing layer and thereby to the attachment flange. In this case, the ring formed connection 412 is provided outside the inner periphery of the flange member 414. The bag 416 is provided in the style of a stoma pouch. Two layers of foil material are welded together to form a pouch. An opening 417 is formed in one of the layers of foil material to provide an entrance to the interior of the pouch. Around the opening, a flat, but flexible surface 418 is provided. This flexible surface 418 is provided with an adhesive 420.

In this embodiment, the distance D6 between the attachment flange and the bag engaging portion is zero, since the flexible flange of the bag engaging portion is directly connected to the attachment flange via an annular connection.

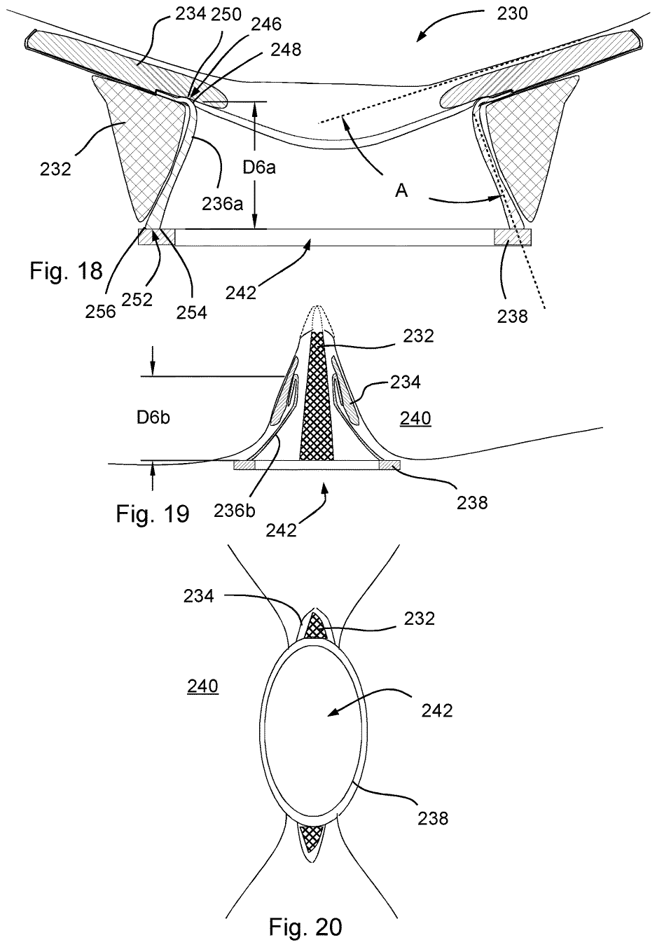

FIGS. 18-20 show schematic illustrations of an embodiment of an attachment member 230 suitable for the system of the first invention.

The embodiment of FIGS. 18-20 is similar in principle to the embodiment of FIG. 11, however a holding element 232 is provided at the forward and rearward portions of the attachment flange 234. The holding element is a thin piece of material which is stiffer than the tubular portion 236 and which ensures that the bag engagement member 238 is held in a relatively constant position relative to the anus of the user. The bag engagement member is arranged such that the opening of the bag engagement member is essentially centered with regards to the anus. The tubular portion is more flexible and can collapse inwardly when the sides of the buttocks press in on the tubular member. However, when a bowel movement happens, the faecal matter will be pushed out through the anus and out through the buttock cheeks 240 and will push out through the opening 242 of the bag engagement member and into the collection bag.

In this case, the diameter of the tubular portion 236 is larger than the diameters of the tubular conduit solutions previously provided by the applicant. Typical diameters of the tubular portion will be between 40 mm and 50 mm. Likewise, the holding element ensures that the distance between the attachment flange and the bag engaging portion is low, but that the bag engagement portion is held outside the buttocks of the user.

It can also been seen in this embodiment, that the length of the ventral/dorsal portion 236a of the tubular portion on the sagittal plane is given by D6a and the length of the side portion 236b of the tubular portion on a plane which is perpendicular to the sagittal plane and parallel to a centre vector passing through the tubular portion is given by D6b. It can be seen from the figure, that the dimension D6b is less than the dimension D6a. In this way, the side portion of the tubular portion is “shorter” than the dorsal/ventral portions of the tubular portion. Due to this geometry, the attachment flange will fit better to the user and a more secure seal will be provided. Simultaneously, this arrangement also allows the flexible flange of the bag engaging portion to be more “flat” or 2D in the dorsal and ventral portions. In this way, a more secure connection can be provided between the collection bag and the bag engaging portion.

It should be noted that this geometry, can be used in other embodiments disclosed in this specification and others, also without the “holding element”. For example, for the embodiment shown in FIG. 11, only the sagittal plan is shown. However, a section through a coronal plane could disclose similar dimensions of D6a and D6b.

For the sake of understanding the claims, the connection between the tubular portion and the attachment flange is a first ring formed connection 246. One could imagine a cross section taken through the tubular portion, at the exact location where the tubular portion extends out from the attachment flange. This first ring formed connection has an innermost point 248 and an outermost point 250 on both sides of a centre axis of the tubular portion. Likewise, the connection between the tubular portion and the bag engaging portion is a second ring formed connection 252. Again, the second ring formed connection 252 has an innermost point 254 and an outermost point 256. The “length” of the tubular portion can be defined by the distance between the innermost points (248,254) of the first and second ring formed connection, or by the distance between the outermost points (250, 256) of the first and second ring formed connection. In this embodiment, due to the thin wall thickness of the tubular portion, the distance between innermost points and the distance between outermost points is essentially the same.

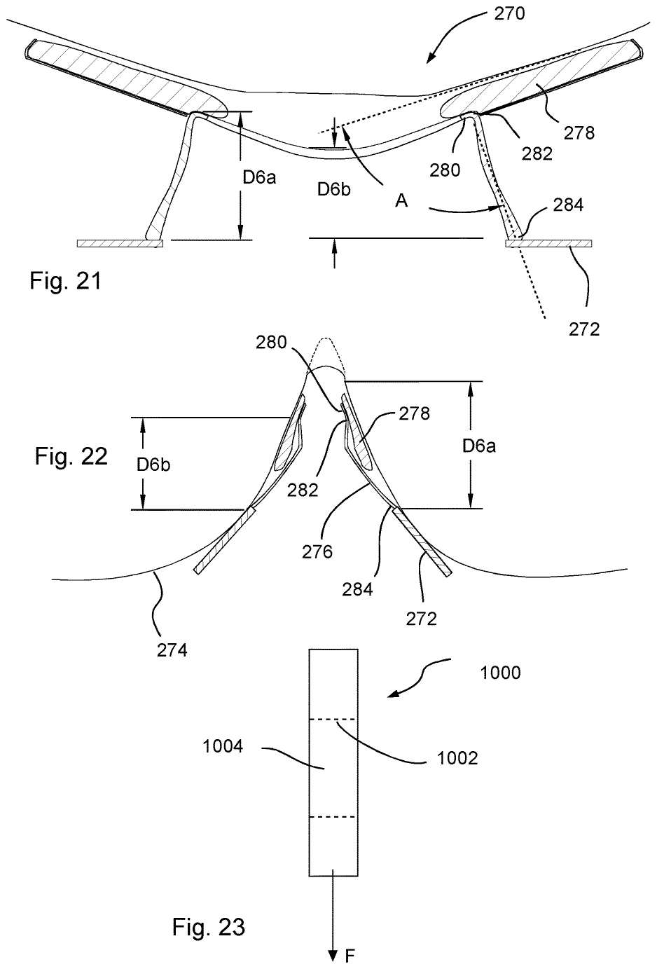

Another embodiment 270 of an attachment member suitable for the system of the first invention having similar geometry, but without the holding element, is illustrated in FIGS. 21 and 22.

This embodiment is very similar to the embodiment of FIGS. 18-20 and as such will not be described in detail. However, it is to be noted that the flexible flange 272 of the bag engaging portion is shown arranged between the buttocks 274 of the user. The flexible flange has been “folded” roughly in half, as can be seen by comparing the position of the flexible flange in the sagittal plane with the position of the flexible flange in the perpendicular plane. Another difference is that the tubular portion 276 is connected to the attachment flange 278 via a thin flange 280 integrally attached to the first end 282 of the tubular portion. The thin flange extends from the tubular portion in towards the opening in the attachment flange, in contrast to the embodiment shown in FIGS. 18-20. This embodiment, is also easier to clean, since the tubular portion tapers outwardly due to the angle A and the inner portion of the flexible flange is roughly the same diameter as the inner diameter of the second end 284 of the tubular portion. Hence, there are no “trapped” areas which are hard to clean.

In this specification, the term “elasticity” or “stretchability of the flange is often used to describe the properties of the flange. The Elasticity/stretchability of the flange portion can be measured as the elongation of a strip 1000 cut out of the flange portion when exposed to a certain load. To be more concrete and to provide actual values for comparison, a strip of 15×70 mm was cut out of some different attachment flanges from body waste collection systems available on the market and two lines 1002 were added to a central portion 1004 of the strip spaced apart by 30 mm. See FIG. 23.

The strip was then hung up and a load was applied. After 1 minute the space between the two lines was measured. The following load samples were used: 50 g, 100 g, 200 g, 300 g and 500 g.

The elongation (E) was recorded as the distance (D) between the two lines at the corresponding weight. For example, the elongation E50 is the elongation for a load of 50 g and is found via the formula:

E50=100*D50/D0

Where D50 is the distance between the two lines with the 50 g load and D0 is the distance between the two lines without any load (3 cm in the current example).

The following samples were tested:
1) FURINE (A sample cut from a flange of Furines External Faecal Catheter)
2) HOLLISTER (A sample cut from a flange of Hollisters Faecal collection Pouch Flextend Incare)
3) COLOPLAST (A sample cut from a flange of Coloplast ostomy pouch (Sensura Mio)
4) A sample of the material used in the attachment flange in some embodiments disclosed in this specification.

It should be noted that samples 1-3 are commercially available products representing the current state of the art in attachment flanges for perianal attachment.

| | | | | |
|---|---|---|---|---|
| Weight (g) | Hollister | Coloplast | Furine | Current |

Distance (cm)

| | | | | |
|---|---|---|---|---|
| 50 | 3.1 | 3.1 | 3.3 | 6.8 |
| 100 | 3.1 | 3.2 | 3.6 | 10.3 |
| 200 | 3.2 | 3.2 | 3.6 | 10.3 |
| 300 | 3.2 | 3.4 | 5.5 | 18 |
| 500 | 3.5 | 4.1 | 7.3 | 22 |

Elongation %

| | | | | |
|---|---|---|---|---|
| 50 | 103% | 103% | 110% | 227% |
| 100 | 103% | 107% | 120% | 343% |
| 200 | 107% | 107% | 143% | 483% |
| 300 | 107% | 113% | 183% | 600% |
| E500 | 117% | 137% | 243% | 733% |

As seen from the table, the Faecal Collection Pouch from Hollister is relatively limited in elongation, while Coloplast's ostomy pouch also shows relatively low elongation.

In contrast, the material used in the attachment flange according to some embodiments disclosed in this specification has more than 700% elongation at 500 gram per 15 mm of a sample strip. While this provides a very comfortable and effective fit, lower elongations will also work. However, it has been found, that in contrast to currently available products, more elastic attachment flanges do provide a much more effective attachment to the peri-anal skin.

It is to be noted that the figures and the above description have shown the example embodiments of the different inventions in a simple and schematic manner. Many of the specific mechanical details have not been shown since the person skilled in the art should be familiar with these details and they would just unnecessarily complicate this description. For example, the specific materials used and the specific fabrication techniques have not been described in detail since it is maintained that the person skilled in the art would be able to find suitable materials and suitable processes to manufacture the products according to the current inventions.

1. A system for collecting body waste from an anus of a user, said system comprising:
a. an attachment member arranged to be attached to the perianal skin of a user, said attachment member comprising:
i. an attachment flange which is arranged to be attached to the perianal skin surrounding the anus of the user such that a fluid tight seal is established between the attachment flange and the perianal skin of the user entirely around the anus, said attachment flange comprising a skin friendly adhesive layer with a skin facing surface,
ii. an opening in the attachment flange which is in fluid communication with the anus of the user when the attachment member is attached to the user,
iii. a bag engaging portion comprising a flexible flange and an opening in said flexible flange, said opening in said flexible flange being in fluid communication with the opening in the attachment flange, said flexible flange of said bag engaging portion being arranged to be folded in half due to the press of the buttocks of the user, and
iv. a tubular portion arranged between the attachment flange and the bag engaging portion, said tubular portion providing a fluid connection between the opening in the attachment flange and the opening of the bag engaging portion, a first end of the tubular portion being connected to the attachment flange via a ring formed connection and an outer end of the tubular portion being connected to the flexible flange of the bag engaging portion, and
b. a collection bag, said collection bag comprising
i. a reservoir for collecting and storing faecal matter from the user,
ii. an opening which is in fluid communication with the reservoir, and
iii. a flexible surface surrounding the opening for detachably attaching the flexible surface of the collection bag to the flexible flange of the bag engaging portion of the attachment member via an adhesive, said flexible surface of the collection bag and said bag engaging flange of the attachment member are both roughly folded in half when the bag is attached to the attachment member.

2. A system according to claim 1, characterized in that the tubular portion is arranged such that a dimension D4 of an “opening” vector connecting the outermost points of the ring formed connection between the attachment flange and the tubular portion in a sagittal plane of a user when connected to a user, is greater than 20 mm and less than 80 mm.

3. A system according to claim 2, characterized in that the bag engaging portion, the tubular portion and the attachment flange are arranged such that a dimension D6a, in the sagittal plane of a user when connected to a user, from an outermost portion of the ring formed connection to an outermost portion of the connection between the tubular portion and the bag engaging portion arranged furthest from the ring formed connection along a direction which is perpendicular to an “opening” vector connecting the outermost points of the ring formed connection in a sagittal plane of a user when connected to a user is less than the minimum radial dimension along the surface of the attachment flange from the centre of the opening in the attachment flange to the outer periphery of the attachment flange.

4. A system according to claim 2, characterized in that the bag engaging portion, the tubular portion and the attachment flange are arranged such that a dimension D6a, in the sagittal plane of a user when connected to a user, from an outermost portion of the ring formed connection to an outermost portion of the connection between the tubular portion and the bag engaging portion arranged furthest from the ring formed connection along a direction which is perpendicular to an
“opening” vector connecting the outermost points of the ring formed connection in a sagittal plane of a user when connected to a user is less than 5 cm or less than 3 cm.

5. A system according to claim 1, characterized in that the bag engaging portion, the tubular portion and the attachment flange are arranged such that
a. a dimension D6a, in the sagittal plane of a user when connected to a user, from an outermost portion of the ring formed connection to an outermost portion of the connection between the tubular portion and the bag engaging portion arranged furthest from the ring formed connection along a direction which is perpendicular to an “opening” vector connecting the outermost points of the ring formed connection in a sagittal plane of a user when connected to a user and/or
b. a dimension D6b, in a “perpendicular” plane which is perpendicular to an “opening” vector connecting the outermost points of the ring formed connection in a sagittal plane of a user when connected to a user, from an outermost portion of the ring formed connection to an outermost portion of the connection between the tubular portion and the bag engaging portion arranged furthest from the attachment flange along a direction which is perpendicular to the “opening” vector
c. are/is less than the radial dimension along the surface of the attachment flange from the centre of the opening in the attachment flange to the outer periphery of the attachment flange along said “perpendicular” plane.

6. A system according to claim 1, characterized in that the bag engaging portion, the tubular portion and the attachment flange are arranged such that a dimension D6a, in the sagittal plane of a user when connected to a user, from an outermost portion of the ring formed connection to an outermost portion of the connection between the tubular portion and the bag engaging portion arranged furthest from the ring formed connection along a direction which is perpendicular to an “opening” vector connecting the outermost points of the ring formed
connection in a sagittal plane of a user when connected to a user is greater than a dimension D6b in a plane which is perpendicular to said “opening” vector, from an outermost portion of the ring formed connection to an outermost portion of the connection between the tubular portion and the bag engaging portion arranged furthest from the attachment flange along a direction which is perpendicular to the “opening” vector.

7. A system according to claim 1, characterized in that the attachment member comprises holding means to hold the opening of the bag engaging portion essentially aligned with the opening in the attachment flange.

8. A system according to claim 1, characterized in that the opening of the bag engaging portion is essentially aligned with the opening in the attachment flange.

9. A system according to claim 1, characterized in that the ring formed connection has a minimum internal diameter which is greater than 30 mm.

10. A system according to claim 1, characterized in that the outermost 5 mm of the attachment flange and the outermost 5 mm of the flexible flange of the bag engaging portion are arranged such that the two portions can displace relative to each other.

11. A system according to claim 1, characterized in that the attachment flange has an outer diameter which is larger than an outer diameter of the ring formed connection.

12. A system according to claim 1, characterized in that the flexible flange of the bag engaging portion is displaced from the outer circumference of the attachment flange.

13. A system according to claim 1, characterized in that the attachment flange is made of a stretchable elastic material while the engaging portion of the collection bag is made of an essentially plastic material.

14. A system according to claim 1, characterized in that an angle A between a “skin facing surface” vector which is parallel with the skin facing surface of the adhesive layer of the attachment flange and a “tubular portion” vector which is parallel to an inner surface of the tubular portion is greater than 70 degrees.

15. A system according to claim 1, characterized in that the inner diameter of the opening in the flexible flange of the bag engaging portion is greater than or equal to the inner diameter of the tubular portion at a ring formed connection between the tubular portion and the flexible flange of the bag engaging portion.

16. A system according to claim 1, characterized in that the opening in the attachment flange is oval in shape.

17. A system according to claim 1, characterized in that the attachment flange comprises a first portion having an inner periphery and an outer periphery arranged spaced away from the inner periphery in a direction away from the opening in the attachment flange and a second portion extending between the outer periphery of the first portion and an outer periphery of the second portion which is further away from the opening in the attachment flange than the outer periphery of the first portion, said first portion having an elongation per unit force applied along the attachment flange and said second portion having a different elongation per unit force applied along the attachment flange which is higher than the elongation per unit force of the first portion.

18. A system according to claim 17, characterized in that the elongation per unit force of the second portion is at least 150% I Newton for a 15 mm wide strip of material of the second portion.

19. A system according to claim 17, characterized in that the attachment flange comprises a central portion extending from the inner periphery of the first portion towards the opening in the attachment flange, said central portion having an elongation per unit force applied along the attachment flange which is higher than the elongation per unit force of the first portion.

20. A system according to claim 19, characterized in that the elongation per unit force of the central portion is at least 150% I Newton for a 15 mm wide strip of material of the central portion.
